(19) United States
(12) Patent Application Publication
(10) Pub. No.: US 2002/0090721 A1
(43) Pub. Date: Jul. 11, 2002

(54) Mucin-containing delivery vehicle for the transport of biomolecules

(76) Inventors: Ashok Kumar Shukla, Woodstock, MD (US); Mukta M. Shukla, Woodstock, MD (US); Amita Shukla, Woodstock, MD (US)

Correspondence Address: ASHOK K. SHUKLA, 10316 kingsway court, ellicott city, MD, 21042, US

(21) Appl. No.: 09/754,868

(22) Filed: Jan. 5, 2001

(51) Int. Cl. C12N 5/08; C07K 14/435

(52) U.S. Cl. 435/366; 530/395

(57) ABSTRACT

In this invention we describe a mucin-containing delivery vehicle for the transport of biomolecules. This vehicle is used to carry and deliver biomolecules such as DNA (deoxyribonucleic acid), RNA (ribonucleic acid), PNA (peptide nucleic acid), polynucleotides, proteins, peptides, lipids, glycoproteins, glycolipids, carbohydrates or a combination of these biomolecules into organisms, cells and interstitial spaces in organisms. The delivery vehicle described in the present invention is comprised of a mucin component, which consists of any mucin or mucin-like moieties and of biomolecules of the same type or of different types. The mucin component of the biomolecules transport vehicle serves to enhance the process of biomolecules transport and delivery. The mucin-based delivery vehicle described in the present invention can be used for biochemical, biomedical, therapeutic or other applications including, but not limited to, delivery of DNA, RNA, PNA, polynucleotides and proteins into cells; gene delivery applications; in vivo, ex vivo or in vitro gene therapy; vaccination of organisms; genetic vaccination of organisms; and, delivery of pharmaceutical products containing biomolecules.

### FIELD OF THE INVENTION

In this invention we describe a mucin-containing delivery vehicle for the transport of biomolecules. This vehicle is used to carry and deliver biomolecules such as DNA (deoxyribonucleic acid), RNA (ribonucleic acid), PNA (peptide nucleic acid), polynucleotides, proteins, peptides, lipids, glycoproteins, glycolipids, carbohydrates or a combination of these biomolecules into organisms, cells and interstitial spaces in organisms or tissues. The delivery vehicle described in the present invention is comprised of a mucin component, which consists of any mucin or mucin-like moieties and of biomolecules of the same type or of different types. The mucin component of the biomolecules transport vehicle, as described in the present invention, serves to enhance the process of biomolecules transport and delivery by facilitating the binding of the biomolecules to the delivery vehicle. The vehicle described in the present invention can also contain any other components, such as lipids, proteins or other molecules used as a means of performing the transport and delivery of desired biomolecules.

The mucin-based delivery vehicle described in the present invention can be used for biochemical, biomedical, therapeutic, clinical, diagnostic or other applications in organisms and cells including, but not limited to, delivery of DNA, RNA, PNA, polynucleotides and proteins into cells; gene delivery applications; in vivo gene therapy, ex vivo gene therapy or in vitro gene therapy; vaccination of organisms; genetic vaccination of organisms; clinical and diagnostic kits and testing; and, delivery of pharmaceutical products containing biomolecules, such as biologically active agents, into cells and organisms. Since current biomolecules delivery mechanisms, especially those used in vaccination and gene delivery, present a number of limitations and disadvantages, the present invention offers tremendous potential for providing an effective, new method for biomolecules delivery, particularly gene delivery for gene therapy in organisms such as humans.

### BACKGROUND OF THE INVENTION

Many new fields in biomedicine and biotechnology rely on the effective delivery of biomolecules into biological cells and organisms. In the present invention biomolecules are defined as any biologically active molecules, whether obtained from biological sources or synthesized artificially, which play an essential role in a physical, chemical or biochemical interaction or reaction in a cell or organism. Thus, in addition to encompassing biological molecules such as proteins and nucleic acids, biomolecules, as they are described in the present invention, also encompass synthetically developed molecules with activity in biological systems, such as pharmaceutical drugs. The search for optimal delivery methods for biomolecules continues since most of the currently available methods offer significant limitations in one respect or another, as discussed here.

Gene Therapy Gen

e therapy is the treatment of diseases and disorders in organisms, primarily humans, through the replacement, repair or alteration of defective genes and/or their defective gene products, such as peptides and proteins. Thus, in gene therapy, genes, which are composed of DNA, need to be delivered to cells so that they can subsequently be expressed in cells such that they result in the production of desired proteins and enzymes or in the replacement, restoration or repair of defective genes. The entire fields of gene therapy and gene delivery depend on the availability of a universal gene delivery vector or mechanism that effectively delivers polynucleotides such as DNA and RNA into cells with high specificity and, ideally, no toxicity.

Gene therapy can be performed in vivo (genes are directly inserted into a host organism for delivery to that organism's cells), in vitro (cells are removed from an organism and gene delivery into those cells is performed outside the organism) and ex vivo (cells are removed from an organism, genes are inserted into those cells outside the organism and these cells are then re-introduced into the organism). Current gene delivery methods include calcium phosphate precipitation, the use of cationic lipid-DNA complexes, liposomes, electroporation and the use of viral vectors. Yet, each of these methods offers distinct disadvantages.

Calcium phosphate precipitation does not always result in sufficient levels of gene delivery into cells and offers very low specificity. Cationic lipids, or positively charged lipids, which are combined in a complex with DNA, are often toxic to cells and thus ineffective for in vivo gene therapy. Liposomes, lipid bi-layer vesicles carrying DNA, which are inserted into cells, also potentially present toxicity, in addition to providing low target cell specificity and insufficient levels of DNA delivery in many instances. Electroporation is a method where very high voltage levels are used to transport genes into cells. Since DNA is highly negatively charged, the application of such an electric current allows for the passage of DNA into cells. Yet, this method cannot be used in in vivo gene delivery and at high voltage levels the death rate of cells is significantly high, limiting the scope of this method.

In viral vector transfection, a virus infects cells, delivering the DNA it contains to those cells. Since viruses can be modified and altered to contain foreign genes or genes needed to perform gene therapy, they can be used to transport DNA into specific cells. In fact, under ideal circumstances, viral vectors can be modified such that the virus retains its gene delivery mechanism but the pathogenic components of its genome are removed. Although viral vectors can be used in vivo, one of their main disadvantages is that they can transform in an organism causing potential harmful effects such as an infection or a strong immunogenic response. The potential of creating an immunogenic response has severely limited the efficacy of viral vectors for gene therapy.

Vaccines

The process of vaccination has been used for the prevention and eradication of many diseases including smallpox, polio, typhus, tetanus and hepatitis A and B. In vaccination a vaccine is administered to an organism to prevent, ameliorate or treat a specific disease. A vaccine typically consists of a preparation of attenuated, weakened or killed pathogens, such as bacteria or viruses, or parts of the structure or body of said pathogens. Many currently available vaccines depend on the delivery of the immunity conferring agents, often pathogen protein and peptide fragments, to an organism. Upon administration the pathogen components of the administered vaccine, known as antigens, stimulate a protective immune response in the host organism that serves to protect the host organism from future, more adverse infections by the same pathogen.

An immune response in an organism is typically mediated by many different cellular and cytolytic parts of the host organism's immune system. The two main arms of the immune system are the humoral arm and the cytolytic arm. The humoral arm is mediated by B lymphoid cells, which produce antibodies and release them into the interstitial fluids where they bind to foreign pathogen proteins, thus eradicating them or marking them for destruction by other cells. T lymphoid cells, also known as cytotoxic T cells, mediate the cytolytic arm. When a pathogen or its components enter the host organism's cells, those cells display fragments of the pathogen's proteins on their surface. T cells recognize foreign protein displayed on the organism's cells and act to destroy those cells, thus eliminating the pathogen that has invaded host cells. In addition to these two main arms of the immune system, many other cellular components are involved in generating an immune response.

During vaccination, the immune system of a host organism's body generates an immune response to the introduced pathogen or pathogen components, thus priming the body's immune system. Ideally, the quantity and type of pathogen or pathogen particles introduced into an organism is sufficient to generate an immune response that confers immunity from subsequent infections but insufficient to generate significant symptoms or manifestations of the disease caused by that pathogen. One of the areas of potential improvement for vaccines relies on the development of more effective methods for delivering pathogen proteins and peptides into organisms.

Genetic Vaccines

While currently available vaccination methods and technologies are well-suited for generating immunity against many known pathogens, they are not well-suited for many other applications due to the limitations of the immune response generated by an organism in response to exposure to only fragments of the pathogen's structure or attenuated pathogens. Often, only either the humoral or cytolytic arm of the immune system is activated and long-term or even effective immunity is not conferred. In fact, many diseases including AIDS, malaria, herpes and cancer could potentially be prevented or treated through the use of vaccines, albeit not through existing vaccine technologies.

In order to develop vaccines and treatments for many of the aforementioned diseases, many new vaccine technologies are currently being explored. One of the latest technologies is genetic vaccines. Genetic vaccines are basically gene-based vaccines in which genes coding for specific proteins or structural components of a pathogen are introduced into the cells of a host organism. Once the newly introduced genes enter the host cells, the expression of those genes leads to the production of proteins and other components of the pathogen. Subsequently, the host's immune system responds to the pathogen proteins, generating a protective immune response.

One of the most attractive features of genetic vaccines is that they can be designed such that only specific, desired proteins from the pathogen enter the host. These proteins would be those specific components of the pathogen that the host recognizes as an antigen and targets for destruction by the immune system. Not only does this method ensure that desired pathogen proteins enter the host but it also prevents the introduction of other, potentially harmful components of the pathogen. Furthermore, genetic vaccines can be used effectively to activate both the humoral and cytolytic arms of the immune system. Yet, as with gene therapy, one of the greatest limiting factors with genetic vaccines is the availability of a suitable gene delivery vehicle. Currently many of the same methods are used for genetic vaccines as for gene therapy and, as discussed, each one of these methods offers significant limitations.

Therapeutic Biomolecules Delivery

Many pharmaceutical treatments depend upon the effective delivery of biomolecules such as proteins and peptides into cells. Currently available drug delivery methods are often limited by their efficacy and by their inability to deliver proteins and other biomolecules effectively since these biomolecules are often degraded by the organism before they perform their desired function. Furthermore, current drug delivery methods are also limited by the extent to which specific therapeutic biomolecules can be targeted to enter only specific types of cells. Thus, the field of drug delivery could benefit significantly from a biomolecules delivery vehicle that is effective and specific in delivering specific therapeutic substances to organisms, such as human beings. In fact, the delivery vehicle described in the present invention can also be developed for the delivery of other pharmaceutical products and non-biological drugs, such as drugs composed of chemicals, into organisms.

Mucins for Biomolecules Delivery

Since current biomolecules delivery methods are so limited in their scope, efficacy and utility, there is a strong need for a non-toxic, safe and highly effective method for the delivery of many different types of biomolecules into cells, organisms or interstitial spaces. The biomolecules delivery vehicle described in the present invention solves many of the problems associated with current biomolecules transport and delivery methods and thus provides a valuable tool for delivering biomolecules to cells and organisms for different applications.

Also, since the specificity of currently available biomolecules delivery methods is very limited, there is a strong need for a delivery vehicle that can provide very high specificity for identifying specific cells as the targets of delivery. The mucin-containing delivery vehicle for biomolecules, as described in the present invention, also provides very high specificity and thus uniquely combines many of the advantageous features of an optimal biomolecules delivery vehicle.

Mucins are glycoproteins with a very high molecular weight, up to several million Daltons. Mucins are commonly present in organisms of many different species. In most mammalian species mucins line the oral cavity, esophagus, stomach and intestines and are secreted by glands in other organs such as the eye. Mucins are rapidly secreted by goblet, glandular and other cell types and they serve a number of different functions. For example, in the lungs, mucins bind bacteria and other microorganisms, facilitating their mucociliary clearance.

Glycoproteins are proteins with carbohydrate molecules attached to them. Mucins are typically from 50 to 90 percent carbohydrate by composition and they are generally watersoluble. In mucin, the carbohydrate molecules are attached as chains to the backbone of the proteins. Since carbohydrates are generally linear molecules the resulting structure can be likened to that of a baby bottle brush, with the carbohydrate molecules forming individual prongs radiating from the central protein backbone. The many different types of carbohydrate chains present on the protein backbone of a mucin unit provide the potential for high levels of specificity when targeting specific cell types.

Furthermore, the physical and chemical structures of mucin molecules are well suited for entangling and subsequently transporting and delivering larger biomolecules such as nucleic acids and peptides. In addition, the mucin component of the biomolecules delivery vehicle described in the present invention can be treated with enzymes such as endoglycosidases or exoglycosidases to enhance the biomolecules binding and delivery capabilities of mucin. Furthermore, the presence of a plurality of different types of carbohydrates on the side chains of mucins offers the potential for creating different types of customized mucins, each suited for a specific biomolecules delivery application. Thus, mucins present a very attractive tool for the delivery of biomolecules.

### ADVANTAGES OF THE INVENTION

Mucin's physical, chemical and biological properties make it a very favorable candidate for biomolecules transport and delivery. In the present invention we describe a biomolecules delivery vehicle that is comprised of the desired biomolecules and one or more different types of mucin, thus drawing upon the favorable properties of mucin that make it an optimal method for transporting biomolecules. Mucins are particularly advantageous for biomolecules delivery because they:

can maintain the stability of biomolecules during the transportation and delivery process;

are capable of delivering large, complex biomolecules, such as nucleic acids to cells;

can be very specifically modified for recognition by specific cells;

can provide targeted delivery to specific cell types;

do not present the potential for generating an immune response as viral vectors for gene therapy do;

can be derived directly from an organism and used for biomolecules delivery in the same or a different organism;

are degraded once they enter desired cells and do not present the same toxicity risks such as cationic lipids in gene therapy;

are well-suited for in vivo, in vitro and ex vitro biomolecules delivery;

can be produced large-scale for many different biomedical, therapeutic and other applications.

The various features of novelty, which characterize the present invention, are pointed out with particularity in the claims annexed to and forming a part of this disclosure. For a better understanding of the invention, its advantages and objects, reference is made to the accompanying drawings and descriptive matter in which a preferred embodiment of the invention is illustrated.

### BRIEF DESCRIPTION OF THE DRAWINGS

The foregoing and still other objects of this invention will become apparent, along with various advantages and features of novelty residing in the present embodiments, from study of the following drawings, in which:

FIG. 1 is an expanded view of one embodiment of a biomolecules transport vehicle comprising mucin and biomolecules, according to the present invention.

FIG. 2 is an expanded view of one embodiment of a biomolecules transport vehicle comprising mucin and DNA, according to the present invention.

FIG. 3 is an expanded view of one embodiment of a biomolecules transport vehicle comprising mucin and biomolecules, in which the carbohydrate chains of mucin are treated with an enzyme, according to the present invention.

FIG. 4 is an expanded view of one embodiment of a sialic acid molecule from the carbohydrate side chains of mucin, where the carboxyl group of sialic acid has been modified, according to the present invention.

FIG. 5 is an expanded view of one embodiment of a sialic acid molecule from the carbohydrate side chains of mucin, where the N-acetyl group of sialic acid has been modified, according to the present invention.

FIG. 6 is an expanded view of one embodiment of a biomolecules transport vehicle comprising mucin, DNA and a liposome, according to the present invention.

FIG. 7 is an expanded view of one embodiment of a biomolecules transport vehicle comprising mucin and biomolecules, in which said vehicle is inside a cell, according to the present invention.

### DESCRIPTION OF THE PREFERRED EMBODIMENTS

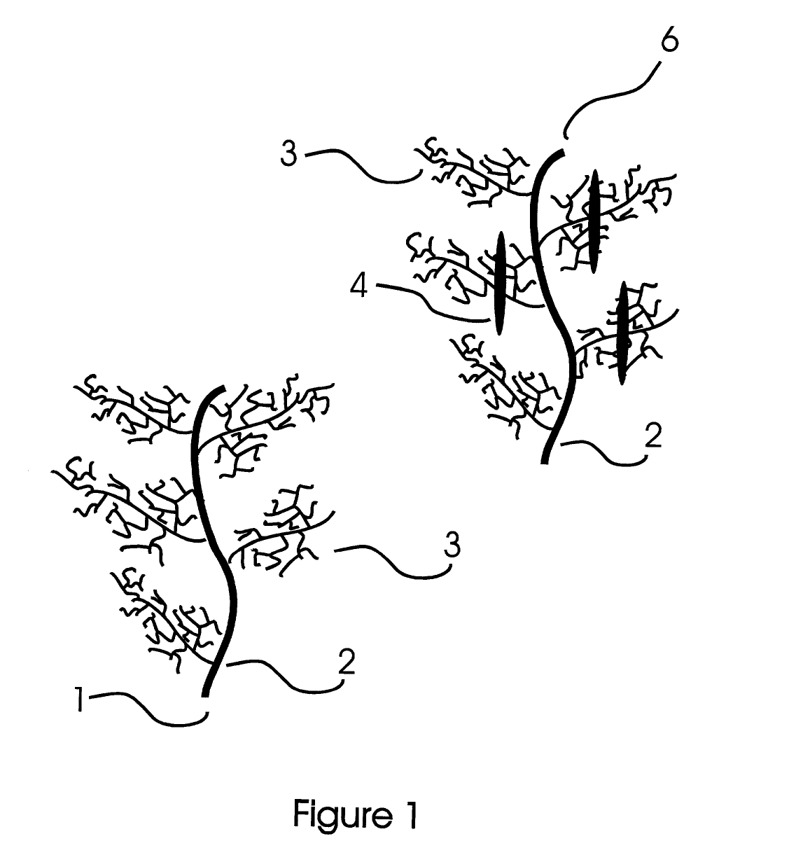

FIG. 1 shows one embodiment of a biomolecules delivery vehicle, according to the present invention. As is shown in FIG. 1, mucin (1) consists of a protein backbone (2) with side chains (3), comprising of carbohydrates, attached to the backbone (2). As is also shown in FIG. 1, to form the delivery vehicle for biomolecules (4), the biomolecules (4) are entangled within the side chains (3) and protein backbone (2) of the mucin molecule (1). Thus, when biomolecules and mucin are entangled together they form a delivery vehicle that can be used to transport the biomolecules into a cell, organism or interstitial spaces in an organism or tissue. In this application, mucin serves as a transporter, carrying the biomolecules and potentially providing cell-specific recognition such that the biomolecules are delivered to specific cells. Cell-specific recognition by mucin is provided by the carbohydrate units on the side chains (3), where selected carbohydrates on the side chains can be added, removed or replaced such that specific cell types recognize specific mucins based on their carbohydrate composition and distribution.

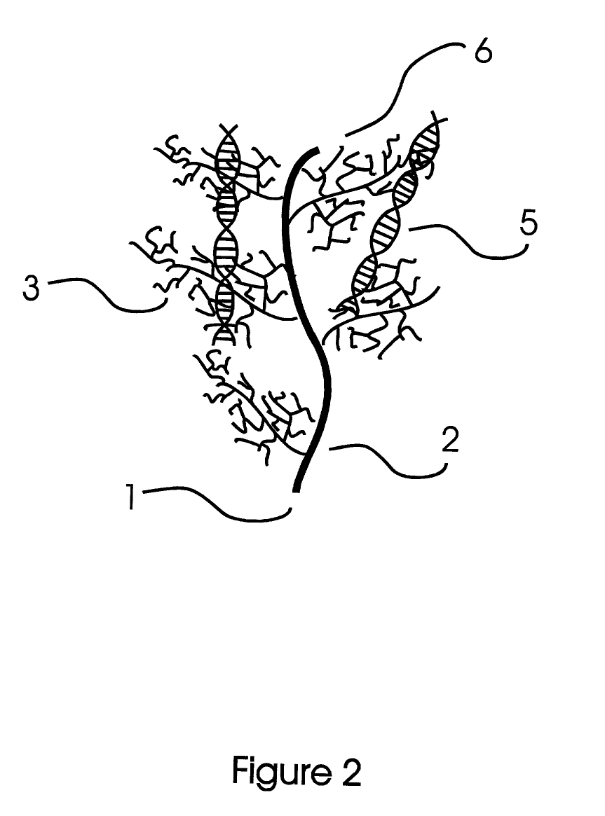

For example, as shown in FIG. 2, mucin (1) can form a complex with and be used to transport DNA (5). As shown in FIG. 2, the strands of the DNA molecules are entangled in the carbohydrate side chains (3) of mucin (1) to form a DNA delivery vehicle. As shown in FIG. 2, the protein backbone (2) and carbohydrate chains (3) of the mucin molecule are intertwined with the strands of the DNA molecule (5). Since both mucin (1) and DNA (5) are large, basically linear molecules, their strands entangle very effectively to create a complex comprising mucin and DNA. When mucin and DNA are present in a complex as shown in FIG. 2, the individual strands of the respective molecules cannot be separated easily, creating the tangled complex shown in the figure. When precipitating agents such ethanol, tannins or an aqueous solution are used mucin and DNA both precipitate, forming an entangled complex. The resulting DNA delivery vehicle comprising mucin can be re-suspended in solution by agitation, shaking or ultrasonication, and can be re-precipitated again when centrifuged. The DNA delivery vehicle comprising mucin, as shown in FIG. 2, can also be purified through centrifugation and washing with buffer.

Such a mucin-DNA delivery vehicle is especially well suited for gene delivery in gene therapy since mucin can be used to transport specific genes, which are composed of DNA, to specific cells. While FIG. 2 shows a DNA delivery vehicle consisting of only DNA and mucin it is understood that any other components such a proteins, carbohydrates, peptides or lipids can be added to the complex to enhance the effectiveness of the vehicle in delivering DNA and to enhance specificity for targeting specific cells. The same holds true for the complex shown in FIG. 1 between mucin (1) and biomolecules (4).

The biomolecules shown in FIG. 1 can be one or more different types of biomolecules selected from the group comprised of DNA (deoxyribonucleic acid), RNA (ribonucleic acid), PNA (peptide nucleic acid), polynucleotides, nucleic acids, proteins, peptides, lipids, carbohydrates, lipoproteins, glycoproteins, glycolipids, inhibitors, antibodies, antigens, and biologically active chemical compounds. Biologically active chemical compounds, also biomolecules, are defined as compounds that are obtained from biological sources or synthesized artificially and which play an essential role in a physical, chemical or biochemical interaction or reaction.

The biomolecules (4) can consist of one or more different types of biomolecules selected from the group comprised of native biomolecules, biological source derived biomolecules, synthetically created biomolecules, modified biomolecules, physically altered biomolecules, chemically altered biomolecules, enzyme modified biomolecules and purified biomolecules. Basically, the biomolecules can be from a natural state, modified, or created synthetically. For example, RNA and PNA, which resemble DNA in their structure, can also be similarly entangled in the carbohydrate side chains (3) of mucin for the delivery of those biomolecules into cells, tissues or organisms and these biomolecules will form a complex with mucin very similar to that shown in FIG. 2.

One of the main advantages of using mucins, as opposed to other currently available nucleic acid delivery mechanisms and tools, is that mucins can be obtained directly from cells and organisms and they present little or no toxicity to organisms. Furthermore, mucins can provide very high levels of specificity due to their carbohydrate side chains.

The mucin (1) used in the delivery vehicle described in the present invention can be any type of mucin, derived from biological sources such as living organisms, derived from non-biological sources or developed synthetically. The mucin (1) can be comprised of one or more different types of mucin selected from the group comprised of native mucin, biological source derived mucin, synthetically created mucin, modified mucin, physically altered mucin, chemically altered mucin, enzyme modified mucin and purified mucin. The mucin (1) can also consist of one or more different types of mucins derived from different sources.

Once mucin (1) is obtained from a desired source it can be purified by chromatographic methods or by precipitation and re-suspension. Also, mucin (1) can be in its native state, as derived from a given source, or the mucin can be modified using biological, physical, chemical, enzymatic, heat-based, electrical current based, pH based or other means of modification. The mucin can be modified prior to its combination with biomolecules, during its combination with biomolecules or after its combination with biomolecules. Physical modification to mucin can include providing rotational energy to mucin (1) or the biomolecules delivery vehicle (6), as shown in FIG. 1. This rotational energy can serve to provide mucin (1) or the delivery vehicle (6) a more compact shape, thus enhancing binding to biomolecules.

Furthermore, the modifications performed on mucin can be any modifications including the removal, alteration or addition of carbohydrate or protein components in mucin's protein backbone (2) and carbohydrate side chains (3). For example, mucin (1) can be modified by the addition to or removal of different monosaccaride groups from its carbohydrate side chains.

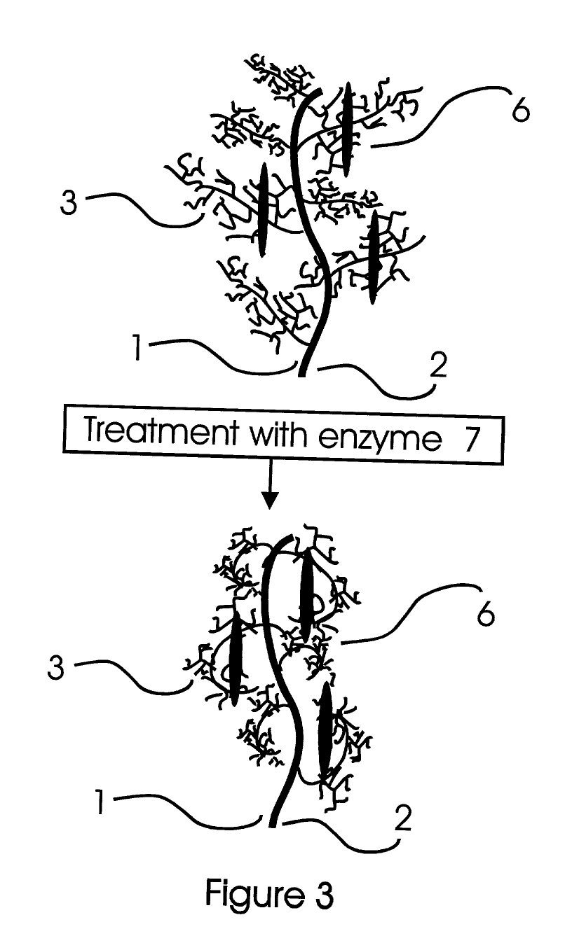

Once the biomolecules (4) are entangled with mucin (1) to form a biomolecules delivery vehicle (6), the vehicle can be modified using biological, chemical, enzymatic, heat-based, electrical current based or other means of modification. For example, as shown in FIG. 3, mucin (1) in the vehicle (6), can be treated with an enzyme (7) such as sialidase. The treatment of the mucin (1) with enzyme (7) results in the alteration of the mucin molecule such that it becomes more effective as a biomolecules delivery vehicle. Mucin, in a native state as shown at the top of FIG. 3, consists of many negatively charged side chains (3), which repel each other to create a structure that resembles a baby bottle brush with individual carbohydrate chains emanating from the protein backbone (2) like prongs.

Once the carbohydrate side chains (3) of mucin (1) are treated with an enzyme, in this instance sialidase, which removes negatively charged sialic acid units from the carbohydrate side chains (3), the individual side chains will fold and entangle the biomolecules and each other more closely, as shown at the bottom of FIG. 3. Thus, as shown in FIG. 3, after treatment with sialidase, the carbohydrate side chains (3) of mucin (1) become more effectively entangled around the biomolecules (4), further enhancing the ability of mucin (1) to bind the biomolecules (4).

In addition the biomolecules delivery vehicle (6) can undergo modifications such as the addition, removal or alteration or carbohydrate or protein components or molecules of said mucin. Furthermore, the biomolecules delivery vehicle (6) can be purified or isolated by any chromatographic or centrifugation methods. The mucin (1) in the vehicle (6) can be modified to target specific cells as the targets of the delivery of the biomolecules (4) carried by the vehicle (6). The specificity of mucin (1) can be controlled through modifications to either the protein backbone (2) or the carbohydrate side chains (3) of mucin.

Most mammalian mucin molecules have sialic acids as terminal molecules. The total or partial removal of sialic acid molecules, either enzymatically or chemically, can further enhance the binding of certain biomolecules to mucin. Carbohydrate molecules can also be selectively added, removed or altered on mucin to change the electrical charge of mucin. For instance, the binding of DNA, which is negatively charged, to mucin is enhanced after the removal of sialic acid from the mucin side chains (3), since sialic acid is also negatively charged. Furthermore, the removal of sialic acid or its charge can also enhance the endocytosis of the biomolecules delivery vehicle, especially when the vehicle is used for the transport of polynucleic acids such as DNA. Endocytosis is the process whereby a cell adheres a certain molecule or complex to its exterior cell membrane and then engulfs it to introduce that molecule or complex into the interior of the cell. For example, when sialic acid is removed from mucin, galactose molecules become the terminal molecules of the mucin carbohydrate chains. Galactose is better recognized by cell surface galactose receptors, thus resulting in more effective endocytosis of the delivery vehicle.

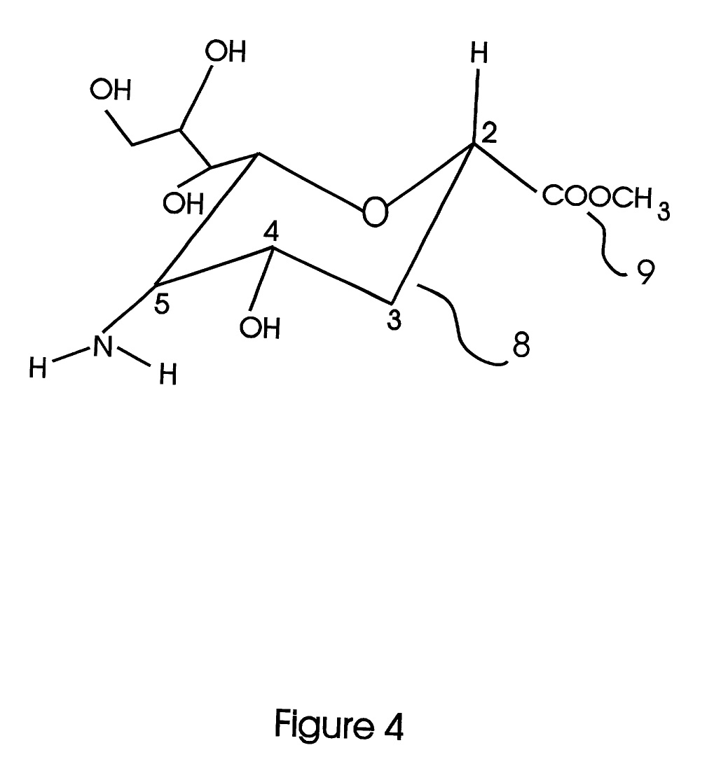

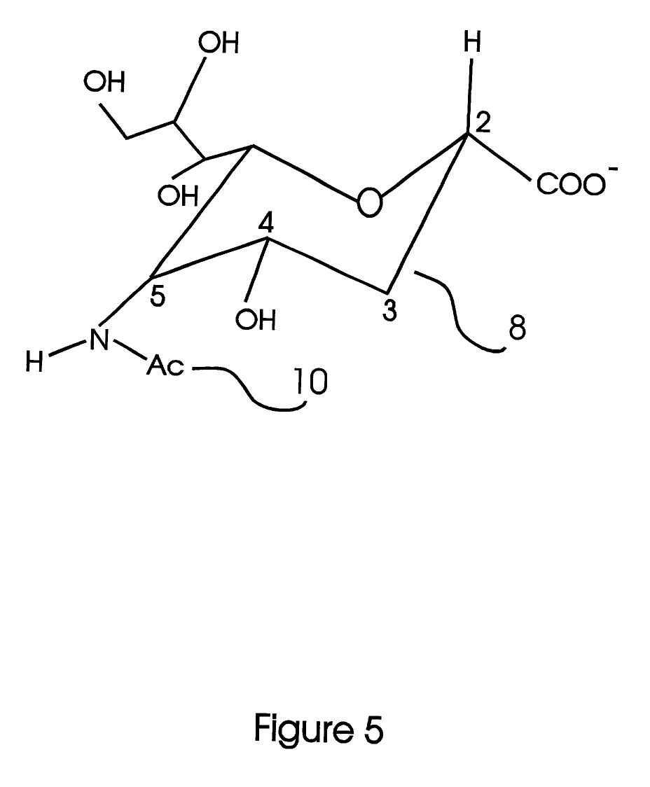

Thus, modifications, such as the removal of sialic acid or its charge, may be advantageous and could be performed on the native mucin to enhance its transport and delivery capabilities. Alternately, as shown in FIG. 4, the negative charges on sialic acid (8) could be suppressed by the esterfication (addition of an ester group) to the carboxyl group (9) of sialic acid (8). The subsequent formation of an ester group (ethyl or methyl) would remove the negative charge from sialic acid. Furthermore, sialic acid has an N-acetyl group at C-5 (10), as shown in FIG. 5. The removal of this acetyl group would confer a positive charge on that component of the sialic acid molecule (9), thus increasing mucin's binding to biomolecules such as negatively charged DNA. Either one or both of these modifications can be performed on sialic acid to enhance the binding of DNA to mucin to form a biomolecules delivery vehicle comprising mucin and DNA.

Furthermore, specific exoglycosidases and endoglycosidases can be used to expose specific carbohydrate groups on the mucin carbohydrate chains (3). This method can be used to tailor the properties of the delivery vehicle to the receptors present on specific target cells and to thus enhance endocytosis and delivery of biomolecules to cells. For examples, lung cells recognize mannose in the terminal position of carbohydrate chains whereas the liver's Kuffer cells recognize galactose in the terminal position. Still other cells may have sialic acid binding protein receptors (sialolectins) for optimal binding and engulfment of the biomolecules delivery vehicle.

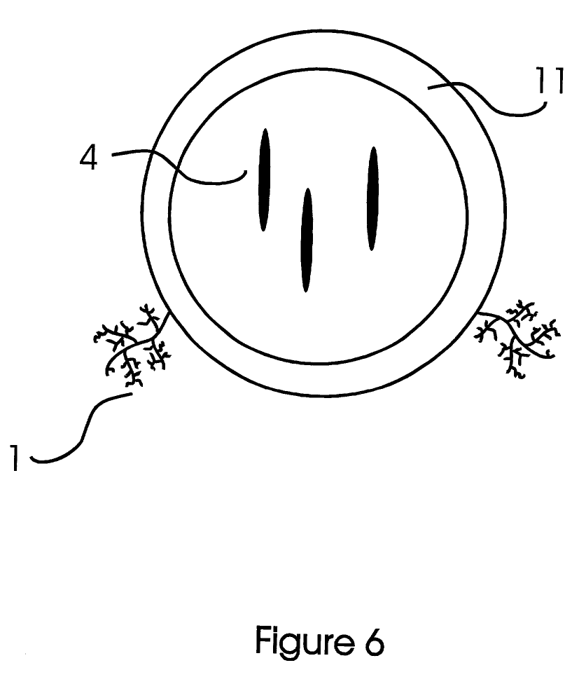

While FIGS. 1 and 2 show mucin (1) forming an entangled complex to comprise the biomolecules delivery vehicle (6), mucin can also form part of a delivery vehicle without being in direct contact with the biomolecules (4). For example, as shown in FIG. 6, the biomolecules (4) can be encapsulated in a lipid vesicle, a liposome (11), and the mucin molecules (1) can be present on the exterior of the liposome (11), as shown. In this delivery vehicle, mucin (1), while not in direct contact with the biomolecules (4), still plays an important role in biomolecules delivery by enhancing the transport of the vehicle and providing specificity for cell recognition.

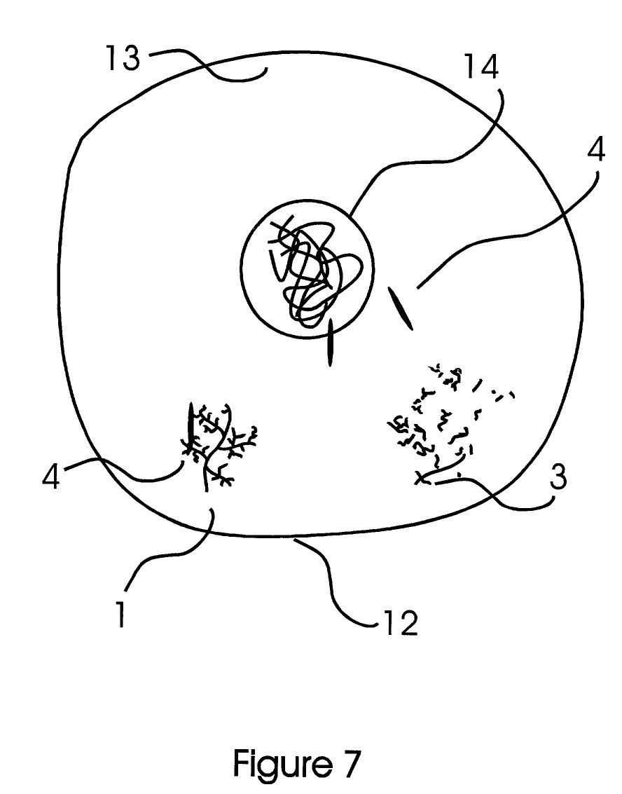

FIG. 7 shows a biological cell (12) that has been entered by a plurality of biomolecules delivery vehicles (6). As shown in FIG. 7, the mucin molecule components, the protein backbone (2) and carbohydrate side chains (3) are broken down upon entry into the cell by enzymes present in the cell's cytoplasm (13). For example, enzymes such as proteases break down proteins and peptides while enzymes such as endoglycosidases and exoglycosidases break down carbohydrate chains into smaller molecules. The biomolecules (4) remain in the cytoplasm (13) of the cell or enter its nucleus (14), depending on the types of biomolecules introduced into the cell and their specificity.

The delivery vehicle described in the present invention can be used to transport biomolecules to specific cell types, into the interstitial spaces of cells or tissues and into any type of cell or organism. The cells used for targets of the delivery vehicle in the present invention can be cells selected from the group consisting of skin, brain, lung, liver, spleen, blood, mucus, muscle, bone, bone marrow, thymus, heart, lymph, cartilage, pancreas, kidney, gall bladder, liver, stomach, intestine, testis, ovary, uterus, breast, rectum, nervous system, eye, gland, lymph node, connective tissue, skeletal system, nervous system, reproductive system, cardiovascular system, digestive system, immune system, urinary system, lymphatic system, and respiratory system cells. The delivery vehicle (6) can be transported into cells, organisms or interstitial spaces in organisms or tissues using means including, but not limited to, means selected from the group consisting of delivering said biomolecules subcutaneously, intradermally, intramuscularly, subdermally, intrathecally, transdermally, intravenously, orally, through inhalation, through insufflation, ocularly, rectally, vaginally, and into the interstitial spaces of tissues.

The mucin-containing delivery vehicle for the transport of biomolecules, as described in the present invention, thus offers a new tool for the transfection of cells and for the in vivo, ex vivo or in vitro, delivery of DNA, RNA, proteins and other biomolecules into cells. The delivery vehicle for the transport of biomolecules, as described in the present invention, can be used for many different applications including but not limited to applications selected from the group consisting of in vivo gene delivery, ex vivo gene delivery, in vitro gene delivery, gene therapy, vaccination, genetic vaccination, drug delivery, therapeutic agents delivery, pharmaceutical products delivery, protein delivery, peptide delivery, enzyme delivery, cell repair, gene repair, DNA repair, cell modification, cell function restoration, gene expression, clinical testing and diagnostic testing.

Furthermore, the ability of mucin to bind and entrap biomolecules can also have other applications such as the development of binding assays and diagnostic kits or tests used to analyze the binding of specific biomolecules or chemical entities to other molecules. This technology can also be used in developing testing and laboratory kits for identifying the presence of certain biomolecules in organisms such as for urine, blood or other bodily secretion diagnostic assays for humans.

The broader usefulness of the present invention may be illustrated by the following example.

### EXAMPLE 1

### Formation of a DNA Delivery Vehicle Containing Mucin

Fluorescence tagged DNA was added to a mucin solution and the mixture was agitated by the use of a vortex for 1-2 minutes. The mucin was precipitated by the addition of isopropanol. The resulting precipitate showed fluorescence whereas the remaining solution showed no fluorescence, indicating that the entire DNA had combined with the mucin to form a mucin-DNA complex.

While a specific embodiment of the invention has been shown and described in detail to illustrate the application of the principles of the invention, it is understood that the invention may be embodied otherwise without departing from such principles and that various modifications, alternate constructions, and equivalents will occur to those skilled in the area given the benefit of this disclosure and the embodiment described herein, as defined by the appended claims.

What is claimed is:

1. A vehicle for the means of transporting biomolecules into biological cells wherein said vehicle is comprised of mucin and biomolecules.

2. Cells of claim 1 wherein said cells are part of a cell group selected from the group comprised of tissues, organs, and organisms.

3. Cells of claim 1 wherein said cells are human cells.

4. Cells of claim 1 wherein said cells are selected from the group comprised of skin, brain, lung, liver, spleen, blood, mucus, muscle, bone, bone marrow, thymus, heart, lymph, cartilage, pancreas, kidney, gall bladder, liver, stomach, intestine, testis, ovary, uterus, breast, rectum, nervous system, eye, gland, lymph node, connective tissue, skeletal system, nervous system, reproductive system, cardiovascular system, digestive system, immune system, urinary system, lymphatic system, and respiratory system cells.

5. Mucin of claim 1 wherein said mucin is a combination of one or more different types of mucin selected from the group comprised of native mucin, biological source derived mucin, synthetically created mucin, modified mucin, physically altered mucin, chemically altered mucin, enzyme modified mucin and purified mucin.

6. Mucin of claim 1 wherein said mucin is in its native state.

7. Mucin of claim 1 wherein said mucin is modified using biological, physical, chemical, enzymatic, heat-based, electrical current based, pH based or other means of modification.

8. Mucin of claim 1 wherein the electrical charge on said mucin is altered to enhance the binding of said mucin to said biomolecules.

9. Mucin of claim 1 wherein said mucin undergoes modifications including, but not limited to, the addition, removal and alteration of carbohydrate or protein components in said mucin.

10. Mucin of claim 1 wherein said mucin contains sialic acid groups.

11. Biomolecules of claim 1 wherein said biomolecules are one or more different biomolecules selected from the group comprised of deoxyribonucleic acid, ribonucleic acid, peptide nucleic acid, polynucleotides, nucleic acids, proteins, peptides, lipids, carbohydrates, lipoproteins, glycoproteins, glycolipids, inhibitors, antibodies, antigens, and biologically active chemical compounds.

12. Biomolecules of claim 1 wherein said biomolecules are one or more different types of biomolecules selected from the group comprised of native biomolecules, biological source derived biomolecules, synthetically created biomolecules, modified biomolecules, physically altered biomolecules, chemically altered biomolecules, enzyme modified biomolecules and purified biomolecules.

13. The vehicle of claim 1 wherein said vehicle is modified using biological, physical, chemical, enzymatic, heat-based, electrical current based, pH based or other means of modification.

14. The vehicle of claim 1 wherein said vehicle undergoes modifications including, but not limited to, the addition, removal or alteration of carbohydrate or protein components or molecules of said mucin.

15. The vehicle of claim 1 wherein said vehicle is purified and isolated by chromatographic methods.

16. The vehicle of claim 1 wherein said vehicle is purified and isolated by centrifugation methods.

17. The vehicle of claim 1 wherein the mucin in said vehicle is modified to deliver said biomolecules to specific target cells.

18. The vehicle of claim 1 wherein said vehicle is used for applications selected from the group consisting of in vivo gene delivery, ex vivo gene delivery, in vitro gene delivery, gene therapy, vaccination, genetic vaccination, drug delivery, therapeutic agents delivery, pharmaceutical products delivery, protein delivery, peptide delivery, enzyme delivery, cell repair, gene repair, DNA repair, cell modification, cell function restoration, gene expression, clinical testing, and diagnostic testing.

19. The means for transporting biomolecules of claim 1 wherein said means is selected from the group consisting of delivering said biomolecules subcutaneously, intradermally, intramuscularly, subdermally, intrathecally, transdermally, intravenously, orally, through inhalation, through insufflation, ocularly, rectally, vaginally, and into the interstitial spaces of tissues.
